(19) United States
(12) Patent Application Publication
(10) Pub. No.: US 2023/0314408 A1
(43) Pub. Date: Oct. 5, 2023

(54) IMMUNE PROFILING AND METHODS OF USING SAME TO PREDICT RESPONSIVENESS TO AN IMMUNOTHERAPY AND TREAT CANCER

(71) Applicant: GoPath Laboratories LLC, Buffalo Grove, IL (US)

(72) Inventors: Sarah S. BACUS, Hinsdale, IL (US); Cristopher A. HAMM, Chicago, IL (US)

(21) Appl. No.: 18/014,053

(22) PCT Filed: Jul. 2, 2021
(86) PCT No.: PCT/US21/40281

§ 371 Date: Dec. 30, 2022

Related U.S. Application Data
(60) Provisional application No. 63/047,305, filed on Jul. 2, 2020.

(51) Int. Cl. G01N 33/50; C07K 16/28; G16H 20/10

(52) U.S. Cl. CPC G01N 33/5011; C07K 16/2896; G16H 20/10

(57) ABSTRACT

The present disclosure provides methods for treating non-small cell lung cancer (NSCLC) in a subject in need thereof by obtaining a biological sample from the NSCLC from the subject; measuring the expression of the following genes associated with immune activation in the biological sample: IFNGR2, MICB, MICA, STATE, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, CCL2, CD28, CD40, OX40, 4-1BB, GITR, CD27, ICOS, CD226, B7, CD226, TCR, and CD40L; measuring the expression of the following genes associated with immune inhibition in the biological sample: ES, CD86, HAVCR2, LAGS, PDCD1, TBX21, TNFRSF14, IDO1, PDCD1LG2, CD47, VTCN1, CD274, MIF, CD276, LGALS3, CTLA4, PD1, TIM3, BTLA, TIGIT, CD96, H3, VISTA, CD112R, and GITR; determining that the NSCLC is responsive to an immunotherapy where the expression of fifteen or more of the genes associated with immune activation are upregulated and ten or less of the genes associated with immune inhibition are upregulated; and administering an immunotherapy to the subject where the subject is determined to be responsive to the immunotherapy.

### CROSS REFERENCE TO RELATED APPLICATION

This PCT application claims priority to U.S. Application Ser. No. 63/047,305, entitled “IMMUNE PROFILING AND METHODS OF USING SAME TO PREDICT RESPONSIVENESS TO AN IMMUNOTHERAPY AND TREAT CANCER,” filed on Jul. 2, 2020, the entirety of which is incorporated herein by reference.

### TECHNICAL FIELD

The present disclosure relates generally to methods of predicting responsiveness to an immunotherapy and using same to treat cancer.

### BACKGROUND

Lung cancer is one of the most common cancers worldwide. With over 1.8 million new cases of diagnosed each year, lung cancer accounts for >1.6 million deaths a year. Approximately 85% of all lung cancers are classified as non-small cell lung cancer (NSCLC). The 5-year survival rate of advanced NSCLC cancer is <20%, but recent advances in targeted therapy and immunotherapy have improved survival rates in subsets of NSCLC patients.

Recent advances in NSCLC immunotherapy are due to the use of immune checkpoint inhibitors, which target the immune inhibitory proteins programmed cell death protein 1 (PD-1) or programmed cell death protein ligand-1 (PD-L1). The FDA recently approved the PD-1 antibodies pembrolizumab (Keytruda) and nivolumab (Opdivo), and the PD-L1 antibody atezolizumab (Tecentriq) for the treatment of NSCLC. Although the PD-1 and PD-L1 antibodies provide an objective response in subsets of patients, only 15-30% of patients respond to individual immune checkpoint inhibitors and responders may ultimately develop therapeutic resistance. While immunotherapy is effective in a subset of patients, other patients may experience treatment-related adverse events, and a small percentage of patients may experience accelerated disease following treatment with an immune checkpoint inhibitor.

PD-L1 expression, as determined by immunohistochemistry, correlates with response to immune checkpoint inhibitors. However, the use of PD-L1 as a standalone predictive biomarker is complicated by the observation that PD-L1 negative tumors may also respond to immune checkpoint inhibitors. As such, there exists a need to determine whether a tumor will or will not respond to treatment with an immune checkpoint inhibitor.

### SUMMARY

The present disclosure solves the above need by predicting whether an NSCLC is responsive or unresponsive to an immunotherapy. Such methods comprise determining where the expression of fifteen or more of the genes associated with immune activation are upregulated and ten or less of the genes associated with immune inhibition are upregulated. A subject predicted to be responsive to the immunotherapy is administrated the immunotherapy while a subject predicted to be unresponsive to the immunotherapy is not administered the immunotherapy.

The present disclosure provides method for treating non-small cell lung cancer (NSCLC) in a subject in need thereof by obtaining a biological sample from the NSCLC from the subject; measuring the expression of the following genes associated with immune activation in the biological sample: IFNGR2, MICB, MICA, STATE, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, CCL2, CD28, CD40, OX40, 4-1BB, GITR, CD27, ICOS, CD226, B7, CD226, TCR, and CD40L; measuring the expression of the following genes associated with immune inhibition in the biological sample: ES, CD86, HAVCR2, LAGS, PDCD1, TBX21, TNFRSF14, IDO1, PDCD1LG2, CD47, VTCN1, CD274, MIF, CD276, LGALS3, CTLA4, PD1, TIM3, BTLA, TIGIT, CD96, H3, VISTA, CD112R, and GITR; determining that the NSCLC is responsive to an immunotherapy where the expression of fifteen or more of the genes associated with immune activation are upregulated and ten or less of the genes associated with immune inhibition are upregulated; and administering an immunotherapy to the subject where the subject is determined to be responsive to the immunotherapy.

In some embodiments of each or any of the above- or below-mentioned embodiments, the immunotherapy is an antibody.

In some embodiments of each or any of the above- or below-mentioned embodiments, the antibody is a monoclonal antibody.

In some embodiments of each or any of the above- or below-mentioned embodiments, the monoclonal antibody is an anti-PD-1 or anti-PD-L1 antibody.

In some embodiments of each or any of the above- or below-mentioned embodiments, the antibody is pembrolizumab, nivolumab, or atezolizumab.

In some embodiments of each or any of the above- or below-mentioned embodiments, the immunotherapy is a small molecule.

In some embodiments of each or any of the above- or below-mentioned embodiments, the biological sample is assayed for genes associated with immune activation and genes associated with immune inhibition by analyzing nucleic acid obtained from the biological sample.

In some embodiments of each or any of the above- or below-mentioned embodiments, the biological sample is assayed for genes associated with immune activation and genes associated with immune inhibition by analyzing protein obtained from the biological sample.

In some embodiments of each or any of the above- or below-mentioned embodiments, the biological sample is from a tumor biopsy.

In some embodiments of each or any of the above- or below-mentioned embodiments, the biological sample is from an aspirate.

The present disclosure also provides methods for conducting a clinical trial by selecting subjects for the clinical trial that are responsive to an immunotherapy by obtaining a biological sample from the NSCLC from the subject; measuring the expression of the following genes associated with immune activation in the biological sample: IFNGR2, MICB, MICA, STATE, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, CCL2, CD28, CD40, OX40, 4-1BB, GITR, CD27, ICOS, CD226, B7, CD226, TCR, and CD40L; measuring the expression of the following genes associated with immune inhibition in the biological sample: ES, CD86, HAVCR2, LAGS, PDCD1, TBX21, TNFRSF14, IDO1, PDCD1LG2, CD47, VTCN1, CD274, MIF, CD276, LGALS3, CTLA4, PD1, TIM3, BTLA, TIGIT, CD96, H3, VISTA, CD112R, and GITR; determining that the NSCLC is responsive to an immunotherapy where the expression of fifteen or more of the genes associated with immune activation are upregulated and ten or less of the genes associated with immune inhibition are upregulated; selecting subjects for inclusion in a clinical trial that are responsive to the immunotherapy; administering an immunotherapy to the subject where the subject is determined to be responsive to the immunotherapy; and seeking regulatory approval for the immunotherapy.

In some embodiments of each or any of the above- or below-mentioned embodiments, immunotherapy is an antibody.

In some embodiments of each or any of the above- or below-mentioned embodiments, the antibody is a monoclonal antibody.

In some embodiments of each or any of the above- or below-mentioned embodiments, the monoclonal antibody is an anti-PD-1 or anti-PD-L1 antibody.

In some embodiments of each or any of the above- or below-mentioned embodiments, the antibody is pembrolizumab, nivolumab, or atezolizumab.

In some embodiments of each or any of the above- or below-mentioned embodiments, the immunotherapy is a small molecule.

In some embodiments of each or any of the above- or below-mentioned embodiments, the biological sample is assayed for genes associated with immune activation and genes associated with immune inhibition by analyzing nucleic acid obtained from the biological sample.

In some embodiments of each or any of the above- or below-mentioned embodiments, the biological sample is assayed for genes associated with immune activation and genes associated with immune inhibition by analyzing protein obtained from the biological sample.

In some embodiments of each or any of the above- or below-mentioned embodiments, the biological sample is from a tumor biopsy.

In some embodiments of each or any of the above- or below-mentioned embodiments, the biological sample is from an aspirate.

The present disclosure also provides methods for processing or analyzing a biological sample from a NSCLC from a subject by (a) sequencing nucleic acid molecules from said sample of the NSCLC to yield data comprising one or more levels of gene expression products in said sample of the NSCLC, which one or more levels of gene expression products correspond to the following genes associated with immune activation: IFNGR2, MICB, MICA, STATE, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, CCL2, CD28, CD40, OX40, 4-1 BB, GITR, CD27, ICOS, CD226, B7, CD226, TCR, and CD40L, and the following genes associated with immune inhibition: ES, CD86, HAVCR2, LAGS, PDCD1, TBX21, TNFRSF14, IDO1, PDCD1LG2, CD47, VTCN1, CD274, MIF, CD276, LGALS3, CTLA4, PD1, TIM3, BTLA, TIGIT, CD96, H3, VISTA, CD112R, and GITR; (b) using an algorithm in a computer to process said data from (a) to generate a classification of said biological sample of NSCLC as responsive or unresponsive to an immunotherapy; and (c) electronically outputting a report that identifies said classification of said sample of NSCLC as responsive or unresponsive to the immunotherapy.

In some embodiments of each or any of the above- or below-mentioned embodiments, the immunotherapy is an antibody.

In some embodiments of each or any of the above- or below-mentioned embodiments, the antibody is a monoclonal antibody.

In some embodiments of each or any of the above- or below-mentioned embodiments, the monoclonal antibody is an anti-PD-1 or anti-PD-L1 antibody.

In some embodiments of each or any of the above- or below-mentioned embodiments, the antibody is pembrolizumab, nivolumab, or atezolizumab.

In some embodiments of each or any of the above- or below-mentioned embodiments, the immunotherapy is a small molecule.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1. NSCLC classified by the amount of CD8+ Positive cells. (A) CD8 IHC was used to characterize NSCLC tissues: low (Left panel), moderate (Center panel), or high (Right panel) levels of CD8+ cells (20×; scoring parameters are provided in Table 1). (B) Summary of CD8 IHC in each NSCLC tissue. H: High, M: Moderate, and L: Low level of CD8+ cells. Pathologist IHC scoring parameters are provided in Table 1. “Area of CD8+ Regions (mm²)” refers to the total slide area of CD8+ regions (mm²) as determined by microscope based image analysis.

FIG. 2. Hierarchical Clustering of genes differentially expressed between NSCLC tissues characterized by Low, Moderate (Table 1), and High (Table 2) levels of CD8+ cells. To be included in this heat map, each gene must be listed in both Table 1 (CD8 low vs CD8 moderate) and Table 2 (CD8 Low vs CD8 high tissue). Hierarchical clustering reveals that CD8 low samples tend to cluster closely together, whereas CD8 high samples tend to cluster closely together. Notice the emergence of a potential gene signature associated with CD8+ levels in NSCLC tissues. On the vertical axis, note that tissue names are following by a “_n_” which indicates that normalized data was used for the heat map. The tissue name also includes either an H, M, or L (H: High, M: Moderate, and L: Low), which indicates the level of CD8 cells as determined in FIG. 1.

FIG. 3. Heat map of genes associated with immune activation (RNA-seq Data). For visualization, Heat Map uses a relative color scheme for each row. The relative color scheme uses the minimum and maximum values in each row to convert expression values to colors. Figure contains a subset of genes that were identified from the literature as having a putative role in immune cell activation. The normalized expression values were clustered using hierarchical clustering (one minus the Pearson correlation). On the vertical axis, note that tissue names are following by a “_n_” which indicates that normalized data was used for the heat map. The tissue name also includes either an H, M, or L (H: High, M: Moderate, and L: Low), which indicates the level of CD8 cells determined in FIG. 1.

FIG. 4. Heat map of genes associated with immune inhibition (RNA-seq Data). For visualization, Heat Map uses a relative color scheme for each row. The relative color scheme uses the minimum and maximum values in each row to convert expression values to colors. Figure contains a subset of genes that were selected from the literature as having a putative role in inhibiting the immune response. The normalized expression values were clustered using hierarchical clustering (one minus the Pearson correlation). On the vertical axis, note that tissue names are following by a “_n_” which indicates that normalized data was used for the heat map. The tissue name also includes either an H, M, or L (H: High, M: Moderate, and L: Low), which indicates the level of CD8 cells as determined in FIG. 1.

FIG. 5. Tissue sections from NS_343 were stained for H&E (Panel A), IDO1 (Panel B), PD-L1 (Panel C) and PD-L2 (Panel D) (20× magnification). Notice the positive staining of the dendritic cells and lymphocytes for IDO1. Note the positivity of the tumor (2+, focal 3+) and the surrounding lymphocytes positive for PD-L1 (PD-L2 50% of tumor at a 2+ positivity). (20× magnification).

FIG. 6. Tissue sections from NS_334 were stained for H&E (Panel A), CD-8 (Few; Panel B), IDO1 (Panel C) and PD-L1 (Panel D). Notice the positive staining of IDO1 tumor cells (3+), positive PD-L1 staining in the tumor (1-2+) and positive staining in the lymphocytes (2+).

FIG. 7. Tissue sections from NS_1331 were stained for H&E (Panel A), CD8 (Moderate: Panel B), IDO1 (Panel C) and PD-L1 (Panel D) (20× magnification). Notice the positive staining of the tumor and lymphocytes surrounding the tumor for PD-L1 (3+ in the tumor). In addition, the lymphocytes are positive for IDO1.

FIG. 8 shows a pathway analysis of differentially expressed genes (CD8 low versus CD8 moderate tissue) (normalized data).

FIG. 9 shows a pathway analysis of differentially expressed genes (CD8 low versus CD8 high tissue) (normalized data).

FIG. 10A-B show genes differentially expressed between CD8 low NSCLC tissues and CD8 moderate NSCLC tissues (normalized data)

FIG. 11A-B show genes differently expressed between CD8 low NSCLC tissues and CD8 high NSCLC tissues (normalized data).

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the claims. Various aspects of the subject technology will now be disclosed according to particular but non-limiting examples. Various embodiments described in the present disclosure may be carried out in different ways and variations, and in accordance with a desired application or implementation.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

The inventors have discovered that NSCLC tumors express unique combinations of genes related to the amount of CD8+ cells, immune activation, and immune inhibition, which may ultimately influence immune escape and response to immune checkpoint therapy. Such gene expression profiles may be used to predict subjects with NSCLC that are responsive to an immunotherapy. Subsequently, such subjects predicted to be responsive to the immunotherapy may be treated with the immunotherapy.

In an embodiment, the subject may be predicted to be responsive to an immunotherapy where the amount of expression of the following genes associated with immune activation: IFNGR2 (e.g., NG_007570.2 RefSeqGene; NM_001329128.1→NP_001316057.1), MICB (e.g., NG_021405.1 RefSeqGene; NM_001289160.1→NP_001276089.1), MICA (e.g., NG_034139.1 RefSeqGene; NM_000247.3→NP_000238.1), STAT6 (e.g., NG_021272.2 RefSeqGene; NM_001178078.2→NP_001171549.1), IFIT1 (e.g., NM_001270927.2→NP_001257856.1), IFIT2 (e.g., NM_001547.5→NP_001538.4), IFIT3 (e.g., NM_001031683.4 NP_001026853.1), IFNGR1 (e.g., NG_007394.1 RefSeqGene; NM_000416.3→NP_000407.1), IL16 (e.g., NG_029933.1 RefSeqGene; NM_001172128.2→NP_001165599.1), STAT4 (e.g., NG_012852.1 RefSeqGene; NM_001243835.2→NP_001230764.1), STATSA (e.g., NM_001288718.1→NP_001275647.1), STAT2 (e.g., NG_046314.1 RefSeqGene; NM_005419.4→NP_005410.1), SOCS5 (e.g., NM_014011.4→NP_054730.1), STAT3 (e.g., NG_007370.1 RefSeqGene; NM_001369512.1→NP_001356441.1), TNFSF4 (e.g., NG_011477.1 RefSeqGene; NM_001297562.2→NP_001284491.1), TNFRSF18 (e.g., NM_004195.3→NP_004186.1), CXCL9 (e.g., NM_002416.3→NP_002407.1), IFNG (e.g., NG_015840.1 RefSeqGene; NM_000619.3→NP_000610.2), STAT1 (e.g., NG_008294.1 RefSeqGene; NM_007315.4→NP_009330.1), TNFRSF9 (e.g., NG_052834.1 RefSeqGene; NM_001561.6→NP_001552.2), CXCL10 (e.g., NM_001565.4→NP_001556.2), SOCS1 (e.g., NM_003745.1→NP_003736.1), TNFRSF4 (e.g., NG_046896.1 RefSeqGene; NM_003327.4→NP_003318.1), SOCS3 (e.g., NG_016851.1 RefSeqGene; NM_001378932.1→NP_001365861.1), and CCL2 (e.g., NG_012123.1 RefSeqGene; NM_002982.4→NP_002973.1) is increased 5%, 10%, 15%, 20%, 25%, 30%, 35%, 40%, 45%, 50%, 55%, 60%, 65%, 70%, 75%, 80%, 85%, 90%, 95% or 100% as compared to a normal tissue (a noncancerous tissue) and the following genes associated with immune inhibition: ES (e.g., NG_021472.2 RefSeqGene; NM_000351.7→NP_000342.3), CD86 (e.g., NG_029928.1 RefSeqGene; NM_001206924.1→NP_001193853.1), HAVCR2 (NG_030444.1 RefSeqGene; NM_032782.5→NP_116171.3), LAG3 (e.g., NM_002286.6→NP_002277.4), PDCD1 (e.g., NG_012110.1 RefSeqGene; NM_005018.3→NP_005009.2), TBX21 (e.g., NG_012166.1 RefSeqGene; NM_013351.2→NP_037483.1), TNFRSF14 (e.g., NG_047096.1 RefSeqGene; NM_001297605.1→NP_001284534.1), IDO1 (e.g., NG_028155.1 RefSeqGene; NM_002164.6→NP_002155.1), PDCD1LG2 (e.g., NM_025239.4→NP_079515.2), CD47 (e.g., NM_001382306.1→NP_001369235.1), VTCN1 (e.g., NM_001253849.1→NP_001240778.1), CD274 (e.g., NM_001267706.1→NP_001254635.1), MIF (e.g., NG_012099.1 RefSeqGene; NM_002415.2→NP_002406.1), CD276 (e.g., NG_051242.1 RefSeqGene; NM_001024736.2→NP_001019907.1), and LGALS3 (e.g., NG_017089.1 RefSeqGene; NM_001357678.2→NP_001344607.1) is increased 5%, 10%, 15%, 20%, 25%, 30%, 35%, 40%, 45%, 50%, 55%, 60%, 65%, 70%, 75%, 80%, 85%, 90%, 95% or 100% as compared to a normal tissue (a noncancerous tissue). Alternatively, a subject may be predicted to be responsive to the immunotherapy where the amount of expression of expression of the following genes associated with immune activation: IFNGR2, MICB, MICA, STATE, IFIT1, IFIT2, IFIT3, IFNGR1, IL16, STAT4, STATSA, STAT2, SOCS5, STAT3, TNFSF4, TNFRSF18, CXCL9, IFNG, STAT1, TNFRSF9, CXCL10, SOCS1, TNFRSF4, SOCS3, and CCL2, and the following genes associated with immune inhibition: ES, CD86, HAVCR2, LAG3, PDCD1, TBX21, TNFRSF14, IDO1, PDCD1 LG2, CD47, VTCN1, CD274, MIF, CD276, and LGALS3 is above or below a set threshold.

In an embodiment, a gene associated with immune activation or immune inhibition may be a variant or isoform of any of the genes disclosed herein.

A determination of whether a subject is predicted to be responsive to an immunotherapy may be used to direct a therapeutic regimen for a particular disease or disorder including, for example, cancer such as NSCLC.

The general methods for determining gene expression product levels are known to the art and may include but are not limited to one or more of the following: additional cytological assays, assays for specific proteins or enzyme activities, assays for specific expression products including protein or RNA or specific RNA splice variants, in situ hybridization, whole or partial genome expression analysis, microarray hybridization assays, SAGE, enzyme linked immuno-absorbance assays, mass-spectrometry, immunohistochemistry, or blotting. Gene expression product levels may be normalized to an internal standard such as total mRNA or the expression level of a particular gene including but not limited to glyceraldehyde 3 phosphate dehydrogenase, or tublin.

In an embodiment, gene expression product markers and alternative splicing markers may be determined by microarray analysis using, for example, Affymetrix arrays, cDNA microarrays, oligonucleotide microarrays, spotted microarrays, or other microarray products from Biorad, Agilent, or Eppendorf. Microarrays provide particular advantages because they may contain a large number of genes or alternative splice variants that may be assayed in a single experiment. In some cases, the microarray device may contain the entire human genome or transcriptome or a substantial fraction thereof allowing a comprehensive evaluation of gene expression patterns, genomic sequence, or alternative splicing. Markers may be found using standard molecular biology and microarray analysis techniques as described in Sambrook Molecular Cloning a Laboratory Manual 2001 and Baldi, P., and Hatfield, W. G., DNA Microarrays and Gene Expression 2002.

Microarray analysis begins with extracting and purifying nucleic acid from a biological sample, (e.g. a biopsy or fine needle aspirate) using methods known to the art. For expression and alternative splicing analysis it may be advantageous to extract and/or purify RNA from DNA. It may further be advantageous to extract and/or purify mRNA from other forms of RNA such as tRNA and rRNA.

Purified nucleic acid may further be labeled with a fluorescent, radionuclide, or chemical label such as biotin or digoxin for example by reverse transcription, PCR, ligation, chemical reaction or other techniques. The labeling can be direct or indirect which may further require a coupling stage. The coupling stage can occur before hybridization, for example, using aminoallyl-UTP and NHS amino-reactive dyes (like cyanine dyes) or after, for example, using biotin and labelled streptavidin. The modified nucleotides (e.g. at a 1 aaUTP: 4 TTP ratio) are added enzymatically at a lower rate compared to normal nucleotides, typically resulting in 1 every 60 bases (measured with a spectrophotometer). The aaDNA may then be purified with, for example, a column or a diafiltration device. The aminoallyl group is an amine group on a long linker attached to the nucleobase, which reacts with a reactive label (e.g. a fluorescent dye).

The labeled samples may then be mixed with a hybridization solution which may contain SDS, SSC, dextran sulfate, a blocking agent (such as COT1 DNA, salmon sperm DNA, calf thymum DNA, PolyA or PolyT), Denhardt's solution, formamine, or a combination thereof.

A hybridization probe is a fragment of DNA or RNA of variable length, which is used to detect in DNA or RNA samples the presence of nucleotide sequences (the DNA target) that are complementary to the sequence in the probe. The probe thereby hybridizes to single-stranded nucleic acid (DNA or RNA) whose base sequence allows probe-target base pairing due to complementarity between the probe and target. The labeled probe is first denatured (by heating or under alkaline conditions) into single DNA strands and then hybridized to the target DNA.

To detect hybridization of the probe to its target sequence, the probe is tagged (or labeled) with a molecular marker; commonly used markers are 32P or Digoxigenin, which is non-radioactive antibody-based marker. DNA sequences or RNA transcripts that have moderate to high sequence similarity to the probe are then detected by visualizing the hybridized probe via autoradiography or other imaging techniques. Detection of sequences with moderate or high similarity depends on how stringent the hybridization conditions were applied—high stringency, such as high hybridization temperature and low salt in hybridization buffers, permits only hybridization between nucleic acid sequences that are highly similar, whereas low stringency, such as lower temperature and high salt, allows hybridization when the sequences are less similar. Hybridization probes used in DNA microarrays refer to DNA covalently attached to an inert surface, such as coated glass slides or gene chips, and to which a mobile cDNA target is hybridized.

This mix may then be denatured by heat or chemical means and added to a port in a microarray. The holes may then be sealed and the microarray hybridized, for example, in a hybridization oven, where the microarray is mixed by rotation, or in a mixer. After an overnight hybridization, non specific binding may be washed off (e.g. with SDS and SSC). The microarray may then be dried and scanned in a special machine where a laser excites the dye and a detector measures its emission. The image may be overlaid with a template grid and the intensities of the features (several pixels make a feature) may be quantified.

Various kits can be used for the amplification of nucleic acid and probe generation of the subject methods. Examples of kit that can be used in the present disclosure include but are not limited to Nugen WT-Ovation FFPE kit, cDNA amplification kit with Nugen Exon Module and Frag/Label module. The NuGEN WI-Ovation™ FFPE System V2 is a whole transcriptome amplification system that enables conducting global gene expression analysis on the vast archives of small and degraded RNA derived from FFPE samples. The system is comprised of reagents and a protocol required for amplification of as little as 50 ng of total FFPE RNA. The protocol can be used for qPCR, sample archiving, fragmentation, and labeling. The amplified cDNA can be fragmented and labeled in less than two hours for GeneChip® 3′ expression array analysis using NuGEN's FL-Ovation™ cDNA Biotin Module V2. For analysis using Affymetrix GeneChip® Exon and Gene ST arrays, the amplified cDNA can be used with the WT-Ovation Exon Module, then fragmented and labeled using the FL-Ovation™ cDNA Biotin Module V2. For analysis on Agilent arrays, the amplified cDNA can be fragmented and labeled using NuGEN's FL-Ovation™ cDNA Fluorescent Module.

The raw data may then be normalized, for example, by subtracting the background intensity and then dividing the intensities making either the total intensity of the features on each channel equal or the intensities of a reference gene and then the t-value for all the intensities may be calculated. More sophisticated methods, include z-ratio, loess and lowess regression and RMA (robust multichip analysis) for Affymetrix chips.

It another embodiment, gene expression product levels in an individual may be obtained without first obtaining a sample. For example, gene expression product levels may be determined in vivo, that is in the individual. Methods for determining gene expression product levels in vivo are known to the art and include imaging techniques such as CAT, MRI; NMR; PET; and optical, fluorescence, or biophotonic imaging of protein or RNA levels using antibodies or molecular beacons. Such methods are described in US 2008/0044824, US 2008/0131892, herein incorporated by reference. Additional methods for in vivo molecular profiling are contemplated to be within the scope of the present disclosure.

In an embodiment, molecular profiling includes the step of binding the sample or a portion of the sample to one or more probes of the present disclosure. Suitable probes bind to components of the sample, i.e. gene products, that are to be measured and include but are not limited to antibodies or antibody fragments, aptamers, nucleic acids, and oligonucleotides. The binding of the sample to the probes of the present disclosure represents a transformation of matter from sample to sample bound to one or more probes. The method of diagnosing cancer based on molecular profiling further comprises the steps of detecting gene expression products (i.e. mRNA or protein) and levels of the sample, comparing it to an amount in a normal control sample to determine the differential gene expression product level between the sample and the control; and classifying the test sample by inputting one or more differential gene expression product levels to a trained algorithm of the present disclosure; validating the sample classification using the selection and classification algorithms of the present disclosure; and identifying the sample as positive for a genetic disorder or a type of cancer.

The results of the molecular profiling performed on the sample provided by the individual (test sample) may be compared to a biological sample that is known or suspected to be normal. A normal sample is that which is or is expected to be free of any cancer, disease, or condition, or a sample that would test negative for any cancer disease or condition in the molecular profiling assay. The normal sample may be from a different individual from the individual being tested, or from the same individual. In some cases, the normal sample is a sample obtained from a buccal swab of an individual such as the individual being tested for example. The normal sample may be assayed at the same time, or at a different time from the test sample.

The results of an assay on the test sample may be compared to the results of the same assay on a normal sample. In some cases the results of the assay on the normal sample are from a database, or a reference. In some cases, the results of the assay on the normal sample are a known or generally accepted value by those skilled in the art. In some cases the comparison is qualitative. In other cases the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, gene product expression levels, gene product expression level changes, alternative exon usage, changes in alternative exon usage, protein levels, DNA polymorphisms, coy number variations, indications of the presence or absence of one or more DNA markers or regions, or nucleic acid sequences.

In an embodiment, a specified statistical confidence level may be determined in order to provide a diagnostic confidence level. For example, it may be determined that a confidence level of greater than 90% may be a useful predictor of responsiveness to an immunotherapy. In other embodiments, more or less stringent confidence levels may be chosen. For example, a confidence level of approximately 70%, 75%, 80%, 85%, 90%, 95%, 97.5%, 99%, 99.5%, or 99.9% may be chosen as a useful predictor. The confidence level provided may in some cases be related to the quality of the sample, the quality of the data, the quality of the analysis, and the specific methods used. The specified confidence level for providing a diagnosis may be chosen on the basis of the expected number of false positives or false negatives and/or cost. Methods for choosing parameters for achieving a specified confidence level or for identifying markers with diagnostic power include but are not limited to Receiver Operator Curve analysis (ROC), binomial ROC, principal component analysis, partial least squares analysis, singular value decomposition, least absolute shrinkage and selection operator analysis, least angle regression, and the threshold gradient directed regularization method.

Raw gene expression level data may in some cases be improved through the application of algorithms designed to normalize and or improve the reliability of the data. In some embodiments of the present disclosure the data analysis requires a computer or other device, machine or apparatus for application of the various algorithms described herein due to the large number of individual data points that are processed. A “machine learning algorithm” refers to a computational-based prediction methodology, also known to persons skilled in the art as a “classifier”, employed for characterizing a gene expression profile. The signals corresponding to certain expression levels, which are obtained by, e.g., microarray-based hybridization assays, are typically subjected to the algorithm in order to classify the expression profile. Supervised learning generally involves “training” a classifier to recognize the distinctions among classes and then “testing” the accuracy of the classifier on an independent test set. For new, unknown samples the classifier can be used to predict the class in which the samples belong.

In an embodiment, the robust multi-array Average (RMA) method may be used to normalize the raw data. The RMA method begins by computing background-corrected intensities for each matched cell on a number of microarrays. The background corrected values are restricted to positive values as described by Irizarry et al. Biostatistics 2003 Apr. 4 (2): 249-64. After background correction, the base-2 logarithm of each background corrected matched-cell intensity is then obtained. The back-ground corrected, log-transformed, matched intensity on each microarray is then normalized using the quantile normalization method in which for each input array and each probe expression value, the array percentile probe value is replaced with the average of all array percentile points, this method is more completely described by Bolstad et al. Bioinformatics 2003. Following quantile normalization, the normalized data may then be fit to a linear model to obtain an expression measure for each probe on each microarray. Tukey's median polish algorithm (Tukey, J. W., Exploratory Data Analysis. 1977) may then be used to determine the log-scale expression level for the normalized probe set data.

Methods of data analysis of gene expression levels may further include the use of a feature selection algorithm as provided herein. In some embodiments of the present disclosure, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420).

Methods of data analysis of gene expression levels may further include the use of a pre-classifier algorithm. For example, an algorithm may use a cell-specific molecular fingerprint to pre-classify the samples according to their composition and then apply a correction/normalization factor. This data/information may then be fed in to a final classification algorithm that would incorporate that information to aid in the final diagnosis.

A statistical evaluation of the results of the molecular profiling may provide a quantitative value or values indicative of one or more of the following: the likelihood of diagnostic accuracy, the likelihood of cancer, disease or condition, the likelihood of a particular cancer, disease or condition, the likelihood of the success of a particular therapeutic intervention. Thus a physician, who is not likely to be trained in genetics or molecular biology, need not understand the raw data. Rather, the data is presented directly to the physician in its most useful form to guide patient care. The results of the molecular profiling can be statistically evaluated using a number of methods known to the art including, but not limited to: the students T test, the two sided T test, pearson rank sum analysis, hidden markov model analysis, analysis of q-q plots, principal component analysis, one way ANOVA, two way ANOVA, LIMMA and the like.

In an embodiment, the results of the molecular profiling are presented as a report on a computer screen or as a paper record. In some cases, the report may include, but is not limited to, such information as one or more of the following: the number of genes differentially expressed, the suitability of the original sample, the number of genes showing differential alternative splicing, a diagnosis, a statistical confidence for the diagnosis, the likelihood of cancer or malignancy, and indicated therapies.

In an embodiment, results are classified using a trained algorithm. Trained algorithms of the present disclosure include algorithms that have been developed using a reference set of known malignant, benign, and normal samples. Algorithms suitable for categorization of samples include but are not limited to k-nearest neighbor algorithms, concept vector algorithms, naive bayesian algorithms, neural network algorithms, hidden markov model algorithms, genetic algorithms, and mutual information feature selection algorithms or any combination thereof.

The present disclosure also provides methods for processing or analyzing a biological sample of a subject, comprising: (a) sequencing nucleic acid molecules from said sample of thyroid tissue to yield data comprising one or more levels of gene expression products in said sample of thyroid tissue, which one or more levels of gene expression products correspond to a plurality of genes selected from the group consisting of: IFNGR2, MICB, MICA, STAT6, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, CCL2, ES, CD86, HAVCR2, LAG3, PDCD1, TBX21, TNFRSF14, IDO1, PDCD1LG2, CD47, VTCN1, CD274, MIF, CD276, and LGALS3; (b) using a trained algorithm in a computer to process said data from (a) to generate a classification of said sample as positive or negative for responsiveness to an immunotherapy, wherein said trained algorithm is trained with a plurality of training samples comprising known benign samples and known non-benign samples that is different from said sample of tissue; and (c) electronically outputting a report that identifies said classification of said sample as responsive or unresponsive for said immunotherapy.

In an embodiment, the subject may be predicted to be responsive to an immunotherapy where the amount of expression of the following genes associated with immune activation: IFNGR2, MICB, MICA, STAT6, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, and CCL2 is increased 5%, 10%, 15%, 20%, 25%, 30%, 35%, 40%, 45%, 50%, 55%, 60%, 65%, 70%, 75%, 80%, 85%, 90%, 95% or 100% as compared to a normal tissue (a noncancerous tissue) and the following genes associated with immune inhibition: ES, CD86, HAVCR2, LAG3, PDCD1, TBX21, TNFRSF14, ID01, PDCD1 LG2, CD47, VTCN1, CD274, MIF, CD276, and LGALS3 is increased 5%, 10%, 15%, 20%, 25%, 30%, 35%, 40%, 45%, 50%, 55%, 60%, 65%, 70%, 75%, 80%, 85%, 90%, 95% or 100% as compared to a normal tissue (a noncancerous tissue). Alternatively, a subject may be predicted to be responsive to the immunotherapy where the amount of expression of expression of the following genes associated with immune activation: IFNGR2, MICB, MICA, STAT6, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, and CCL2, and the following genes associated with immune inhibition: ES, CD86, HAVCR2, LAGS, PDCD1, TBX21, TNFRSF14, IDO1, PDCD1 LG2, CD47, VTCN1, CD274, MIF, CD276, and LGALS3 is above or below a set threshold.

The disclosure also provides a method of predicting whether a cancer such as NSCLC will or will not respond to an immunotherapy comprising the steps of: (a) obtaining a biological sample comprising gene expression products; (b) detecting the gene expression products of the biological sample; (c) comparing to an amount in a control sample, an amount of one or more gene expression products in the biological sample to determine the differential gene expression product level between the biological sample and the control sample; (d) classifying the biological sample by inputting the one or more differential gene expression product levels to an algorithm; and (e) identifying the biological sample as positive for a cancer if the algorithm classifies the sample as responsive to the immunotherapy. The biological sample may be obtained from a subject in need thereof such as a subject with NSCLC.

In an embodiment, the difference in gene expression level is at least 10%, 15%, 20%, 25%, 30%, 35%, 40%, 45% or 50% or more. In some embodiments, the difference in gene expression level is at least 2, 3, 4, 5, 6, 7, 8, 9, 10 fold or more.

The disclosure provides a method of screening for gene expression by immunohistochemical or immunocytochemical methods.

Immunohistochemistry (“IHC”) and immunocytochemistry (“ICC”) techniques, for example, may be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically target molecules inside or on the surface of cells. The antibody typically contains a marker that will undergo a biochemical reaction, and thereby experience a change color, upon encountering the targeted molecules. In some instances, signal amplification may be integrated into the particular protocol, wherein a secondary antibody, that includes the marker stain, follows the application of a primary specific antibody.

Immunoshistochemical assays are known to those of skill in the art (e.g., see Jalkanen, et al., J. Cell. Biol. 101:976-985 (1985); Jalkanen, et al., J. Cell. Biol. 105:3087-3096 (1987).

Antibodies, polyclonal or monoclonal, can be purchased from a variety of commercial suppliers, or may be manufactured using well-known methods, e.g., as described in Harlow et al., Antibodies: A Laboratory Manual, 2nd Ed; Cold Spring Harbor Laboratory Press, Cold Spring Harbor, N.Y. (1988). In general, examples of antibodies useful in the present disclosure include anti-phospho-STAT3, anti-phospho-STAT5, and anti-phospho-Akt antibodies. Such antibodies can be purchased, for example, from Upstate Biotechnology (Lake Placid, N.Y.), New England Biolabs (Beverly, Mass.), NeoMarkers (Fremont, Calif.)

Typically, for immunohistochemistry, tissue sections are obtained from a patient and fixed by a suitable fixing agent such as alcohol, acetone, and paraformaldehyde, to which is reacted an antibody. Conventional methods for immunohistochemistry are described in Harlow and Lane (eds) (1988) In “Antibodies A Laboratory Manual”, Cold Spring Harbor Press, Cold Spring Harbor, N.Y.; Ausbel et al (eds) (1987), in Current Protocols In Molecular Biology, John Wiley and Sons (New York, N.Y.). Biological samples appropriate for such detection assays include, but are not limited to, cells, tissue biopsy, whole blood, plasma, serum, sputum, cerebrospinal fluid, breast aspirates, pleural fluid, urine and the like.

For direct labeling techniques, a labeled antibody is utilized. For indirect labeling techniques, the sample is further reacted with a labeled substance.

Alternatively, immunocytochemistry may be utilized. In general, cells are obtained from a patient and fixed by a suitable fixing agent such as alcohol, acetone, and paraformaldehyde, to which is reacted an antibody. Methods of immunocytological staining of human samples is known to those of skill in the art and described, for example, in Brauer et al., 2001 (FASEB J, 15, 2689-2701), Smith-Swintosky et al., 1997.

Immunological methods of the present disclosure are advantageous because they require only small quantities of biological material. Such methods may be done at the cellular level and thereby necessitate a minimum of one cell. Preferably, several cells are obtained from a patient affected with or at risk for developing cancer and assayed according to the methods of the present disclosure.

TABLE 1
IHC Scoring Guidelines
CD8 IHC

| | |
|---|---|
| Rare/Few/Low | 2-5 cells in High Power Field (40×) |
| Moderate | 6-19 cells in High Power Field (40×) |
| High/Abundant | >20 cells in High Power Field |

PD1, PD-L1, and PD-L2

| | |
|---|---|
| Rare/Few/Low | 1-24 cells in High Power Field (40×) |
| Moderate | 25-49 cells in High Power Field (40×) |
| High/Abundant | Above 49 cells in High Power Field (40×) |

pSTAT3, IDO1, and FOXP3

| | |
|---|---|
| Rare/Few/Low | 1+ |
| Moderate | 2+ |
| High/Abundant | 3+ |

CD3 and CD163

| | |
|---|---|
| Rare/Few/Low | 2-5 cells in High Power Field |
| Moderate | 6-19 cells in High Power Field |
| High/Abundant | >20 cells in High Power Field |

### Illustration of Subject Technology as Clauses

Various examples of aspects of the disclosure are described as numbered clauses (1, 2, 3, etc.) for convenience. These are provided as examples, and do not limit the subject technology.

Clause 1. A method for treating non-small cell lung cancer (NSCLC) in a subject in need thereof, the method comprising: obtaining a biological sample from the NSCLC from the subject; measuring the expression of the following genes associated with immune activation in the biological sample: IFNGR2, MICB, MICA, STATE, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, CCL2, CD28, CD40, OX40, 4-1BB, GITR, CD27, ICOS, CD226, B7, CD226, TCR, and CD40L; measuring the expression of the following genes associated with immune inhibition in the biological sample: ES, CD86, HAVCR2, LAGS, PDCD1, TBX21, TNFRSF14, IDO1, PDCD1LG2, CD47, VTCN1, CD274, MIF, CD276, LGALS3, CTLA4, PD1, TIM3, BTLA, TIGIT, CD96, H3, VISTA, CD112R, and GITR; determining that the NSCLC is responsive to an immunotherapy where the expression of fifteen or more of the genes associated with immune activation are upregulated and ten or less of the genes associated with immune inhibition are upregulated; and administering an immunotherapy to the subject where the subject is determined to be responsive to the immunotherapy.

Clause 2. The method of Clause 1, wherein the immunotherapy is an antibody.

Clause 3. The method of Clause 2, wherein the antibody is a monoclonal antibody.

Clause 4. The method of Clause 3, wherein the monoclonal antibody is an anti-PD-1 or anti-PD-L1 antibody.

Clause 5. The method of Clause 4, wherein the antibody is pembrolizumab, nivolumab, or atezolizumab.

Clause 6. The method of Clause 1, wherein the immunotherapy is a small molecule.

Clause 7. The method of Clause 1, wherein the biological sample is assayed for genes associated with immune activation and genes associated with immune inhibition by analyzing nucleic acid obtained from the biological sample.

Clause 8. The method of Clause 1, wherein the biological sample is assayed for genes associated with immune activation and genes associated with immune inhibition by analyzing protein obtained from the biological sample.

Clause 9. The method of Clause 1, wherein the biological sample is from a tumor biopsy.

Clause 10. The method of Clause 1, wherein the biological sample is from an aspirate.

Clause 11. A method for conducting a clinical trial by selecting subjects for the clinical trial that are responsive to an immunotherapy, the method comprising: obtaining a biological sample from the NSCLC from the subject; measuring the expression of the following genes associated with immune activation in the biological sample: IFNGR2, MICB, MICA, STATE, IFIT1, IFIT2, IFIT3, IFNGR1, IL16, STAT4, STATSA, STAT2, SOCS5, STAT3, TNFSF4, TNFRSF18, CXCL9, IFNG, STAT1, TNFRSF9, CXCL10, SOCS1, TNFRSF4, SOCS3, CCL2, CD28, CD40, OX40, 4-1BB, GITR, CD27, ICOS, CD226, B7, CD226, TCR, and CD40L; measuring the expression of the following genes associated with immune inhibition in the biological sample: ES, CD86, HAVCR2, LAGS, PDCD1, TBX21, TNFRSF14, IDO1, PDCD1LG2, CD47, VTCN1, CD274, MIF, CD276, LGALS3, CTLA4, PD1, TIM3, BTLA, TIGIT, CD96, H3, VISTA, CD112R, and GITR; determining that the NSCLC is responsive to an immunotherapy where the expression of fifteen or more of the genes associated with immune activation are upregulated and ten or less of the genes associated with immune inhibition are upregulated; selecting subjects for inclusion in a clinical trial that are responsive to the immunotherapy; administering an immunotherapy to the subject where the subject is determined to be responsive to the immunotherapy; and seeking regulatory approval for the immunotherapy.

Clause 12. The method of Clause 11, wherein the immunotherapy is an antibody.

Clause 13. The method of Clause 12, wherein the antibody is a monoclonal antibody.

Clause 14. The method of Clause 13, wherein the monoclonal antibody is an anti-PD-1 or anti-PD-L1 antibody.

Clause 15. The method of Clause 14, wherein the antibody is pembrolizumab, nivolumab, or atezolizumab.

Clause 16. The method of Clause 11, wherein the immunotherapy is a small molecule.

Clause 17. The method of Clause 11, wherein the biological sample is assayed for genes associated with immune activation and genes associated with immune inhibition by analyzing nucleic acid obtained from the biological sample.

Clause 18. The method of Clause 11, wherein the biological sample is assayed for genes associated with immune activation and genes associated with immune inhibition by analyzing protein obtained from the biological sample.

Clause 19. The method of Clause 11, wherein the biological sample is from a tumor biopsy.

Clause 20. The method of Clause 11, wherein the biological sample is from an aspirate.

Clause 21. A method for processing or analyzing a biological sample from a NSCLC from a subject, comprising: (a) sequencing nucleic acid molecules from said sample of the NSCLC to yield data comprising one or more levels of gene expression products in said sample of the NSCLC, which one or more levels of gene expression products correspond to the following genes associated with immune activation: IFNGR2, MICB, MICA, STATE, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, CCL2, CD28, CD40, OX40, 4-1 BB, GITR, CD27, ICOS, CD226, B7, CD226, TCR, and CD40L, and the following genes associated with immune inhibition: ES, CD86, HAVCR2, LAGS, PDCD1, TBX21, TNFRSF14, IDO1, PDCD1LG2, CD47, VTCN1, CD274, MIF, CD276, LGALS3, CTLA4, PD1, TIM3, BTLA, TIGIT, CD96, H3, VISTA, CD112R, and GITR; (b) using an algorithm in a computer to process said data from (a) to generate a classification of said biological sample of NSCLC as responsive or unresponsive to an immunotherapy; and (c) electronically outputting a report that identifies said classification of said sample of NSCLC as responsive or unresponsive to the immunotherapy.

Clause 22. The method of Clause 21, wherein the immunotherapy is an antibody.

Clause 23. The method of Clause 22, wherein the antibody is a monoclonal antibody.

Clause 24. The method of Clause 23, wherein the monoclonal antibody is an anti-PD-1 or anti-PD-L1 antibody.

Clause 25. The method of Clause 24, wherein the antibody is pembrolizumab, nivolumab, or atezolizumab.

Clause 26. The method of Clause 21, wherein the immunotherapy is a small molecule.

### EXAMPLES

### Example 1: CD8+ Levels in NSCLC

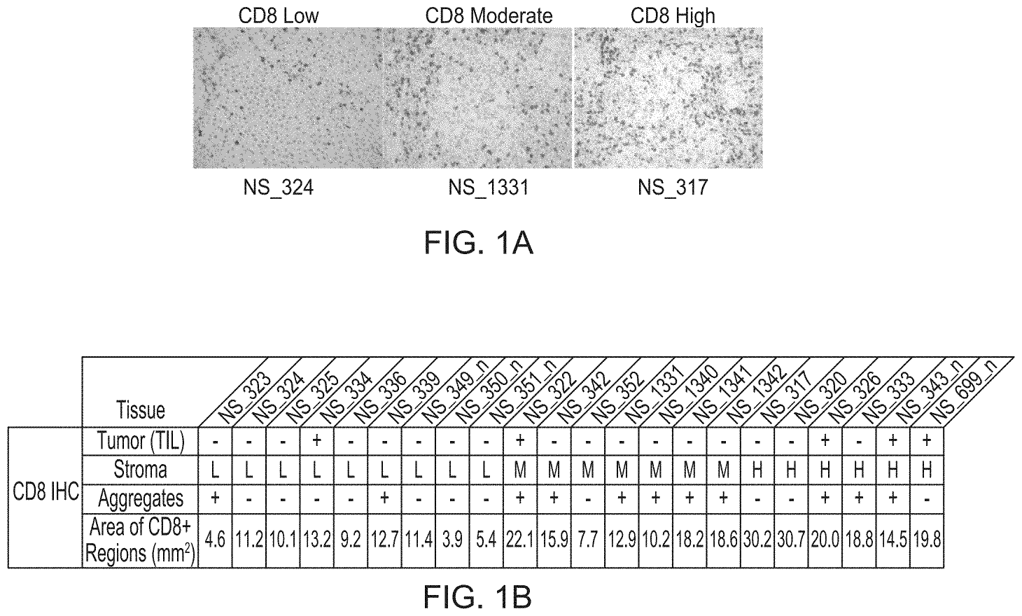

Tumor infiltrating lymphocytes (TILs) are a component of tumor the microenvironment in NSCLC. The TIL levels, subtype, and activation status may influence clinical outcome, and CD8 cell infiltrate density provides information on disease prognosis and responsiveness to immunotherapy. Therefore, IHC was used to classify the amount of CD8+ T-cells in the NSCLC tissues. The amount of CD8+ T-cells varied among NSCLCs, and NSCLCs were classified as having low, moderate, or high levels of CD8+ T-cells (FIG. 1). Of the 22 NSCLC tissues, 9 tissues contained low levels of CD8+ T-cells, 7 tissues contained moderate levels of CD8+ T-cells, and 6 tissues contained high levels of CD8+ T-cells. CD8+ T-cells presented as single cell spreads or as aggregates. CD8+ T-cells aggregate were observed more frequently in the tissues with moderate and high levels of CD8+ T-cells (FIG. 1B). Additional, pathology was confirmed using microscope based image analysis (FIG. 1B).

### Example 2: Immune Gene Expression in NSCLC

CD8 cell levels in the NSCLC tissue may be related to the signaling pathways that regulate the immune response in each tissue. Therefore, a targeted RNA-seq panel was used to profile the expression of genes that may regulate immune cells in NSCLC. The targeted RNA-seq panel consisted of 500 immune related genes. The RNA-seq analysis was performed on 22 NSCLC tissues.

The RNA-seq data was separated into one of three groups based on the level of CD8+ cells in the specific tissue and classified as containing low, moderate, or high levels of CD8+ cells (determined by IHC; FIG. 1). The RNA-seq data revealed gene expression changes between the NSCLC tissues with low, moderate, or high levels of CD8+ cells. Fifty-six genes are significantly differentially expressed between CD8 low NSCLC tissues and CD8 moderate NSCLC tissues (Table 1). In addition, 58 genes are significantly differentially expressed between CD8-low NSCLC tissues and CD8-high NSCLC tissues (Table 2).

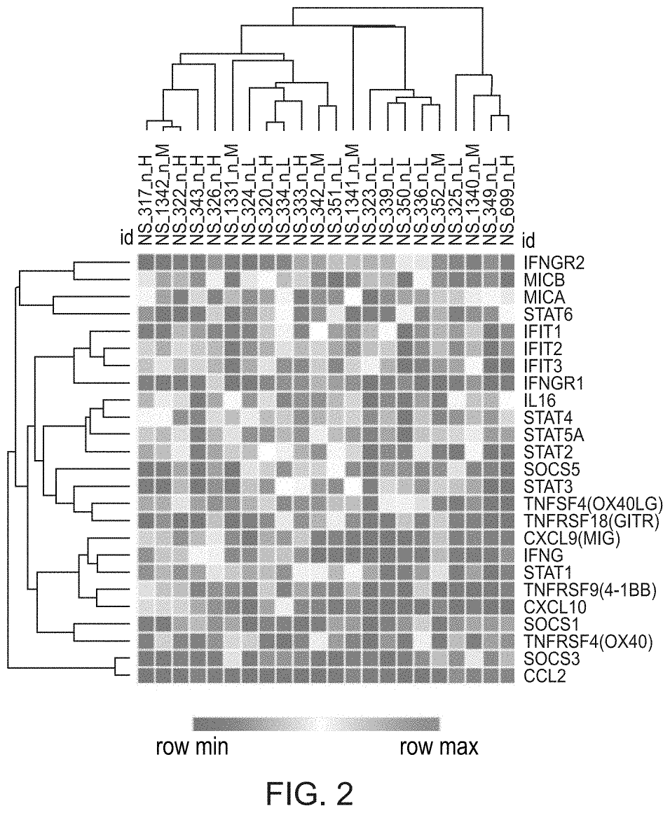

Hierarchical clustering of the differentially expressed genes reveals that NSCLC samples cluster into two overall groups (FIG. 2). CD8-high tissues cluster into one group and CD8-low tissues cluster in another group (FIG. 2). However, CD8-moderate samples cluster among CD8-high samples (4 CD8-moderate tissues, see FIG. 2) and CD8-low tissues (3 CD8-moderate tissues, see FIG. 2). Despite the limited number of NSCLC samples, the clustering reveals a putative gene expression signature that may correlate with tissues that are classified as CD8-high and CD8-low. The clustering analysis also reveals that certain CD8-moderate tissues (classified by CD8 IHC) possess immune gene expression signatures that share a resemblance to expression the profiles of CD8-low tissues or CD8-high tissues. Although classified as CD8-moderate (classified by IHC), the heterogeneity of the CD8-moderate tissues may reflect an immune landscape that lies in-between the CD8-Low and CD8 high tissues, which highlights the importance of combining CD8 IHC with gene expression profiling to attain greater insight into pathways that contribute to the immune response in NSCLC.

Subsequent, pathway analysis revealed that the differentially expressed genes function in immune relevant pathways such as “Allograft Rejection” and “Interferon Gamma Response” (Tables 3 and 4). It is noteworthy that the CD8-high and CD8-moderate tissues express similar gene expression pathways. However, the CD8-high tissues have a greater representation in each pathway (See Supplementary Table S6 and S7 for the specific list of genes that contribute to each pathway map).

Not surprisingly, the CD8-high and CD8-moderate tissues express elevated levels of T cell-related genes (ex. CD8A, GZMA, GZMH, etc.) and chemokines (ex. CXCL9 (MIG), CXCL10, etc.), which may be a component of active immune response in the NSCLC tissues (Tables 1 and 2). Although CD8+ tissues express genes that may be relevant to an active immune environment, it is noteworthy that the same tissues also express inhibitory molecules that may disrupt the anti-tumor immune response. For example, CTLA4,

PDCD1 (PD-1), and TBX21 (T-bet) are expressed at higher levels in the CD8 high tissue than CD8 low tissues (Table 2).

### Example 3. Expression of Genes that Regulate Immune Activation and Immune Suppression

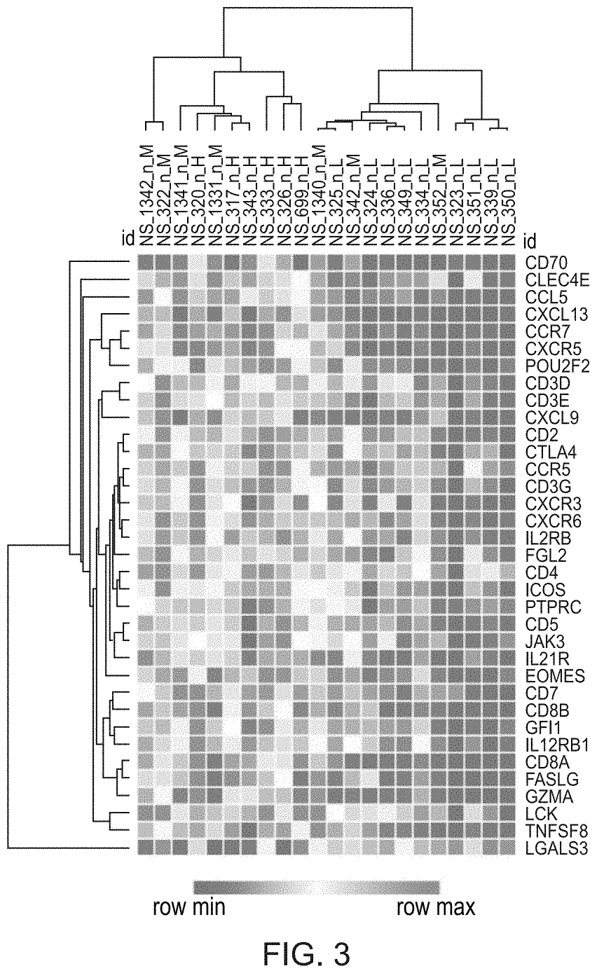

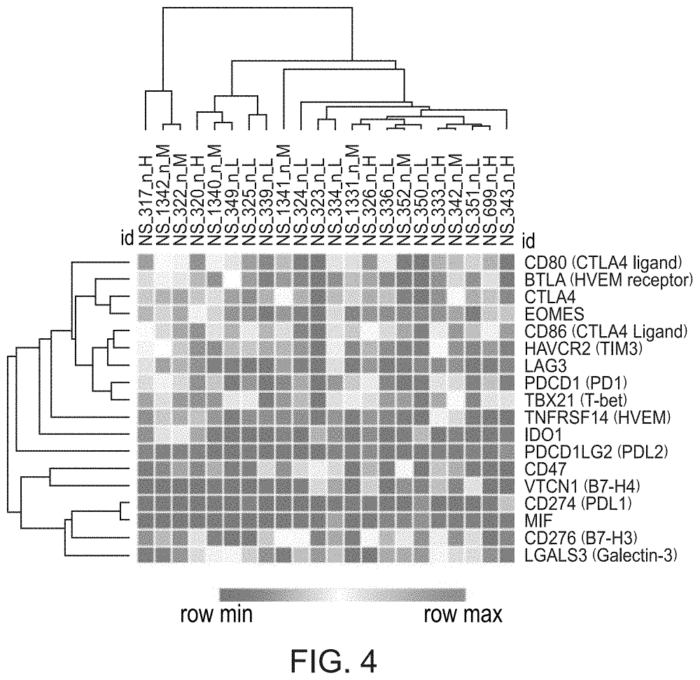

Although the putative CD8 gene signature data provide insight into the genes that are associated the presence of CD8 (CD8 low, moderate, and high) tissues, NSCLCs are known as heterogeneous tumors (Chen et al., 2014). Therefore, the RNA-seq data was used to specifically examine immune-related genes in each NSCLC tissue (immune activation genes: FIG. 3; and immune inhibition genes: FIG. 4). The RNA-seq analysis revealed heterogeneity with respect to genes that play a role in the activation or the inhibition of an anti-tumor immune response (FIGS. 3 and 4). Several tissues express immune activation-related genes (ex. STAT pathway and che-mokines; FIG. 3), whereas several tissues also express immune inhibitory molecules (FIG. 4).

For example, tissues NS_320, NS_326, and NS_333 express multiple genes related to both immune activation signaling and immune inhibitory signaling (FIGS. 3 and 4), and these samples are also characterized by high levels of CD8 cells (FIG. 1B). Alternatively, tissue NS_351 expresses low levels of immune activation signaling and immune inhibitory signaling (FIGS. 3 and 4; NS_351), which correlates with the IHC observation that tissue NS_351 contains a low level of CD8 cells.

Additionally tissues NS_343 and NS_350 both express genes associated with immune activation (FIG. 3). However, tissue NS_343 expresses the inhibitory molecules PD-L1, PD-L2 (PDCD1 LG2), and IDO1 (FIG. 4). Whereas NS_350 expresses high levels of CD274 (PD-L1), IDO1, MIF, CD276 (B7-H3), and LGALS3 (Galectin-3) (FIG. 4).

The expression of the immune checkpoint molecule PD-L1 varies among NSCLC tissues, with highest levels of PD-L1 detected in samples NS_350, NS_343, NS_325, and NS_326. The expression of IDO1 (a gene that may promote immune tolerance to tumor antigens) also varied among tumor tissues. Tissues NS_317 and NS_334 expressed the highest levels of IDO1, whereas tissues NS_336 and NS_351 express low levels of IDO1.

Additionally, CD47, an inhibitor of macrophage phagocytosis, is expressed at lower levels throughout most tissues, but CD47 expression is elevated in NS_1341 and NS_334. LAG3, a negative regulator of T cell activation is expressed at the highest levels in NS_320, _NS_322, and NS_317. The T-cell exhaustion markers T-bet (TBX21) and EOMES are also expressed in several tissues, albeit at low levels. The expression of T-bet and EOMES may indicate the presence of exhausted T-cells, which may have limited ability to respond to an immune checkpoint inhibitor.

Finally, while the NSCLC RNA-seq data reveals some heterogeneity among immune activation and immune inhibitory genes, it is noteworthy that the hierarchical clustering reveals subsets of NSCLC that express similar combinations of genes (FIGS. 3 and 4). For example, tissues NS_317, NS_1342, and NS_322 cluster together in heat maps (FIGS. 3 and 4) and these tissues simultaneously express genes that function in immune activation and immune inhibition.

### Example 4. IHC Confirms the Simultaneous Expression of Multiple Immune Inhibitory Proteins

The RNA-seq data provides insight into the genes and pathways that are expressed in NSCLC, but it is also important to determine if the immune pathways are also active on the protein level. In addition to the CD8 analysis (FIG. 1), IHC was used to measure the expression of the following immune-related genes: CD3, CD163, pSTAT3 (phosphorylated STAT3), FOXOP3, PD1, PD-L1 (CD274), PDL2 (PDCD1), and IDO1. IHC scoring parameters are listed in Table 1. Of the genes with both RNA-seq data and IHC data, all NSCLC samples with protein expression by IHC were also shown to express the corresponding RNA. Genes with moderate to high expression by RNA-seq correlated with the protein levels identified by IHC (CD8, PD-L1, PD-L2, and IDO1). PD-1 and FOXP3 were also detected by both RNA-seq and IHC, but direct correlation with protein expression should be carefully considered as these genes are expressed at relatively low levels in the RNA-seq data. In the RNA-seq data, PD-1 and Foxp3 genes are expressed at <100 transcript counts in a given tissue.

Overall, the level of protein expression varies by tissue and IHC target, however, the most abundantly expressed genes, assessed by RNA-seq, are also expressed at high levels in the IHC analysis. PD-L1 RNA is expressed at highest levels in tissues NS_343 and NS_350, and the IHC confirms that both tissues express high levels of PD-L1 in the NSCLC tumor (FIG. 4). While tissues NS_343 and NS_350 express high levels of PD-L1, it is noteworthy that NS_343 contains a high level of CD8 cells whereas NS_350 contains low levels of CD8 cells. Despite similar PD-L1 levels in NS_343 and NS_350, the tissues are characterized by different patterns of immune-related gene expression (FIGS. 2, 3 and 4).

The IHC also provides biologically relevant information regarding protein localization and distribution. For example, the transcription factor STAT3 was detected in the RNA-seq analysis, but the IHC analysis reveals that phosphorylated-STAT3 (pSTAT3) is expressed in both tumor cells and stromal cells. In this current IHC analysis, pSTAT3 levels do not directly correlate with the level of CD8 cells, and pSTAT3 levels vary between tissues. However, STAT3 signaling may influence tumorigenesis and the immune response, and therefore pSTAT3 expression data my provide insight into the nature of the anti-tumor immune response in the tumor and stroma of a given NSCLC tissue.

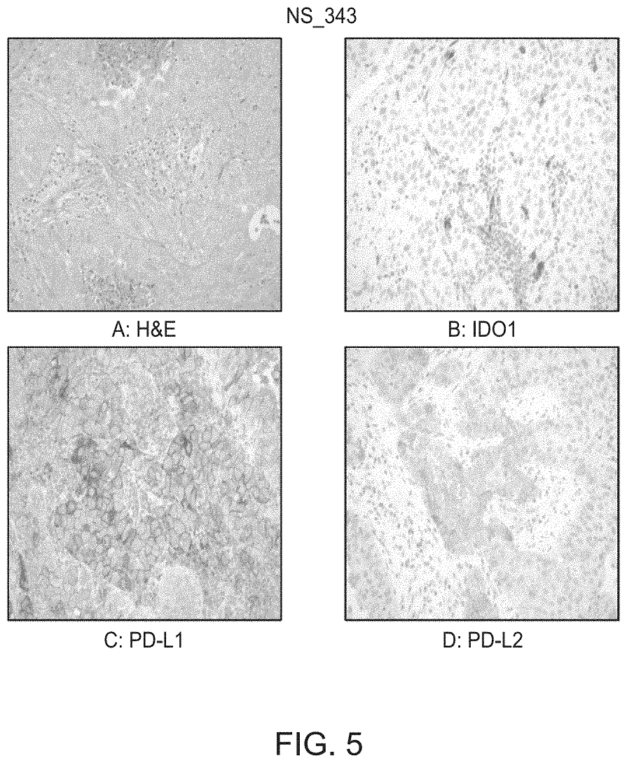

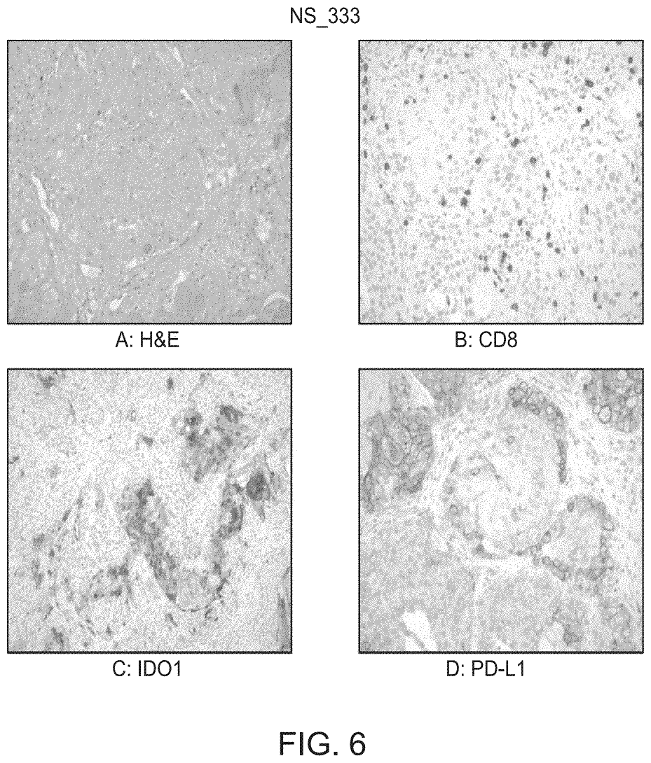

The variable expression of CD8 (T-cell marker) and CD163 (macrophage marker) reveals the diverse nature of the immune response in NSCLC tissues. The diversity of the immune response is also revealed by the observation that many of the NSCLC tissues express more than one immune inhibitory protein. For example, tissue NS_343 expresses both PD-L1 and PD-L2 (FIG. 5). Several NSCLC tissues also express the immune inhibitory proteins PD-L1 and IDO1. Tissues NS_317, NS_1342, and NS_334 express moderate (or high) levels of PDL1 and IDO1 in tumor cells (FIG. 6). Interestingly, tumors NS_317 and NS_1342 are also characterized by the presence of high levels of stromal CD8+ lymphocytes, but no CD8+ lymphocytes are detected within the tumor area.

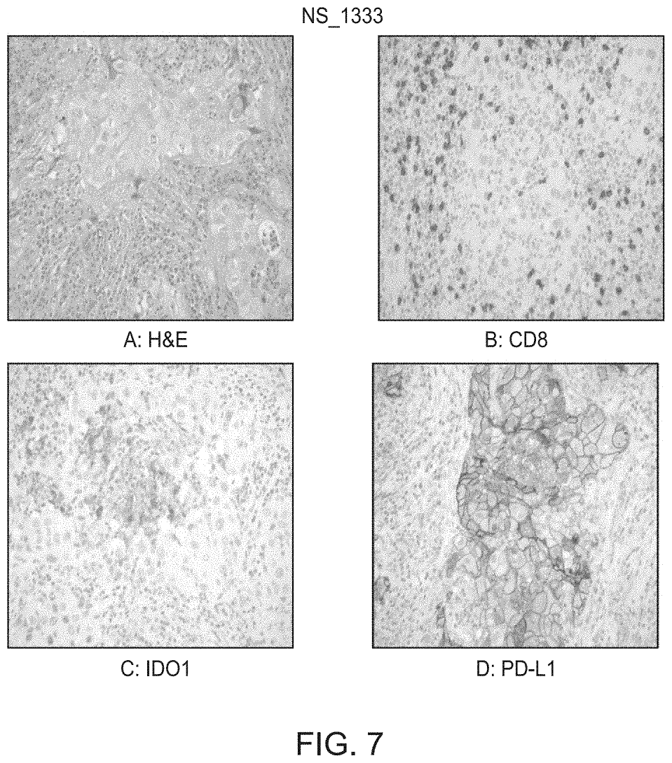

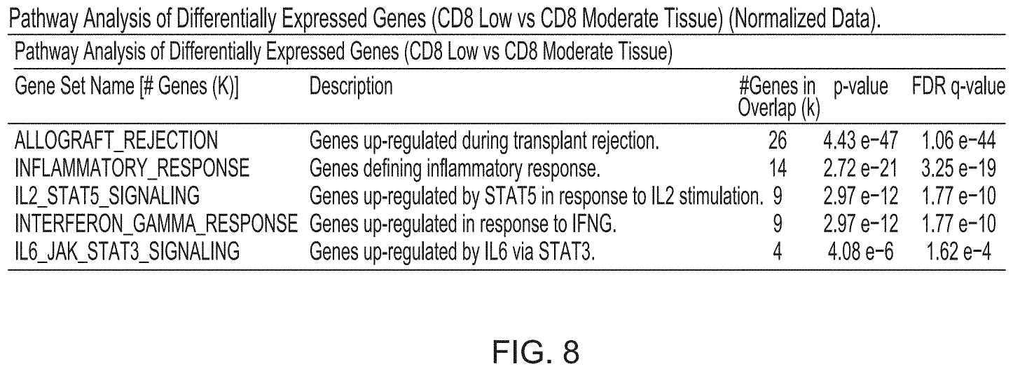

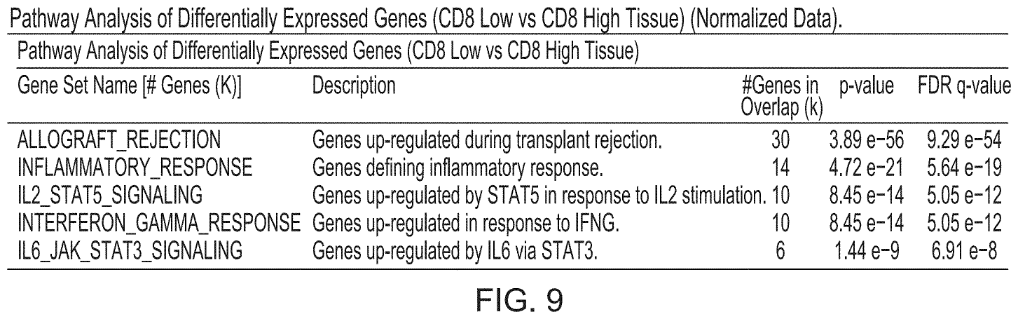

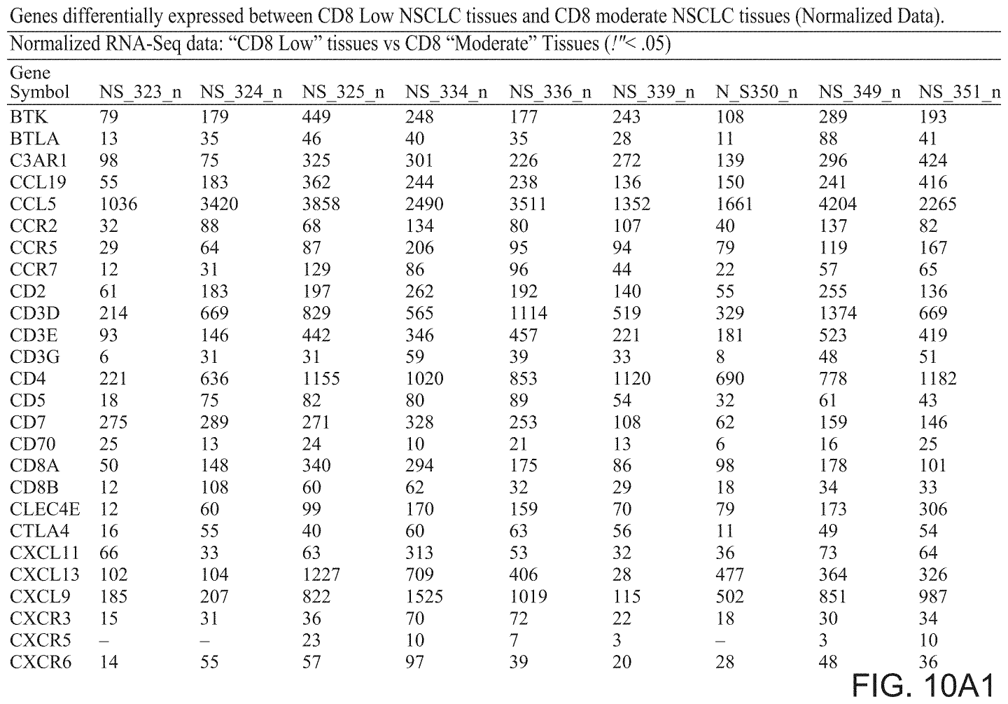

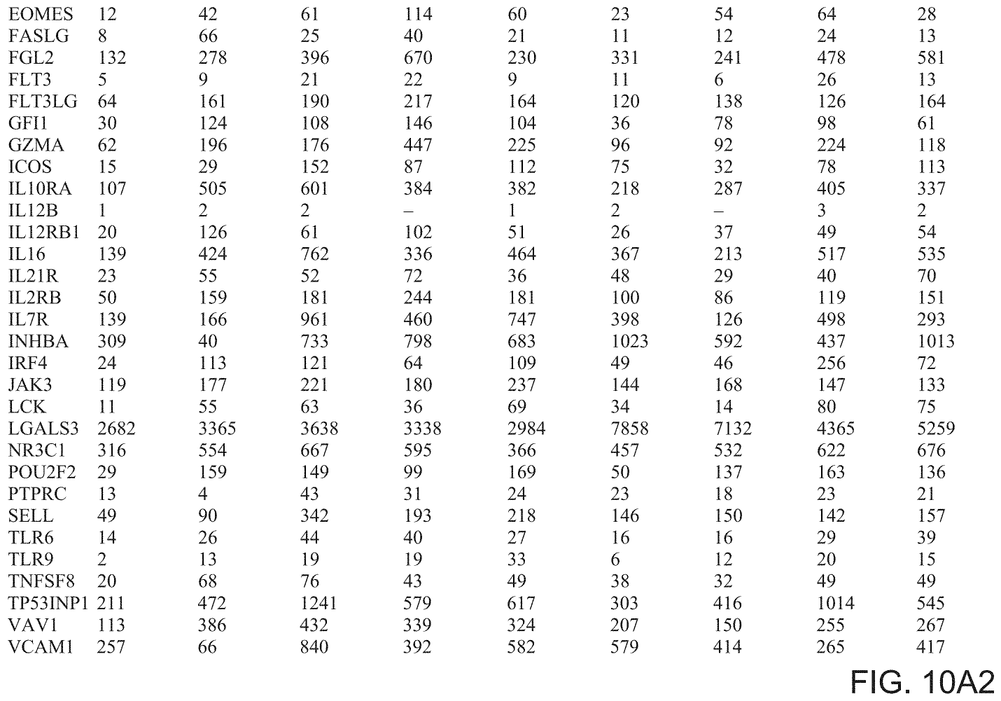

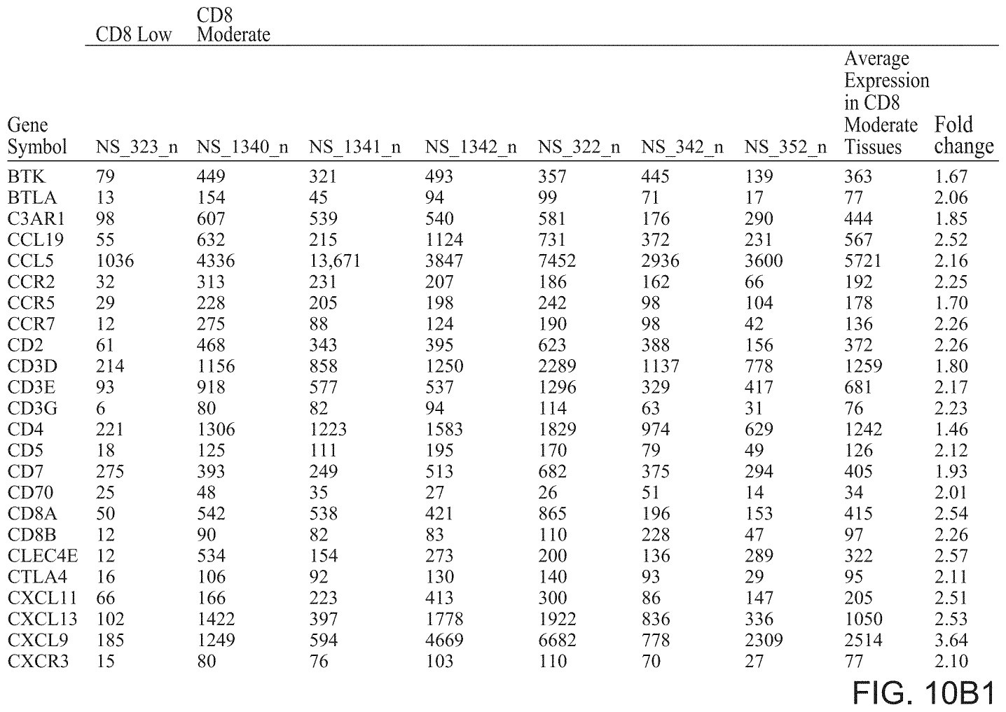

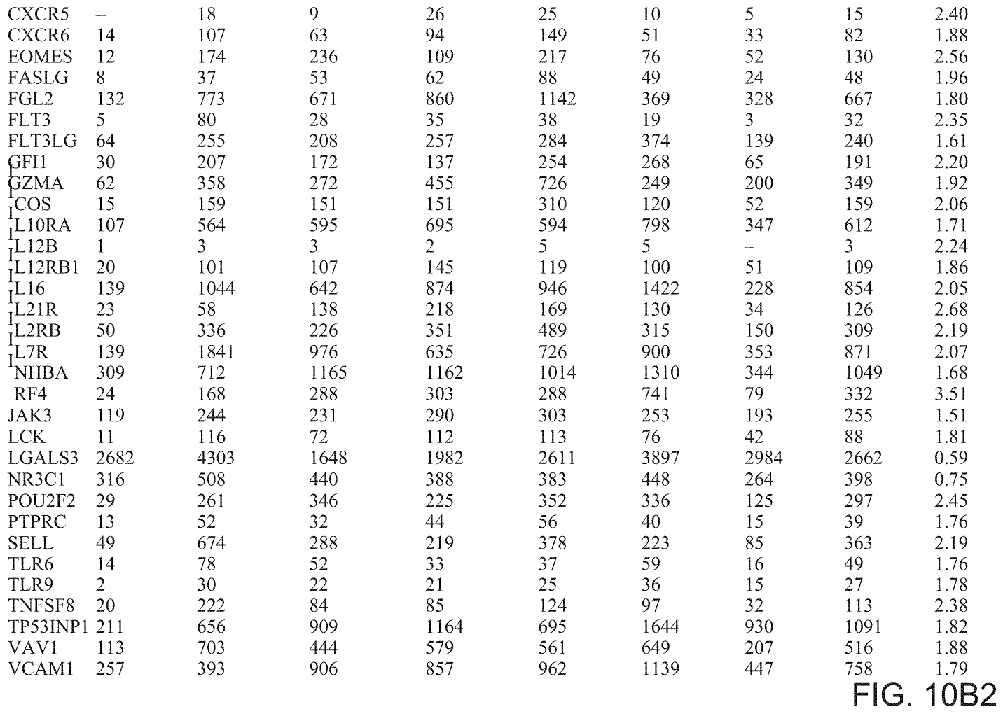

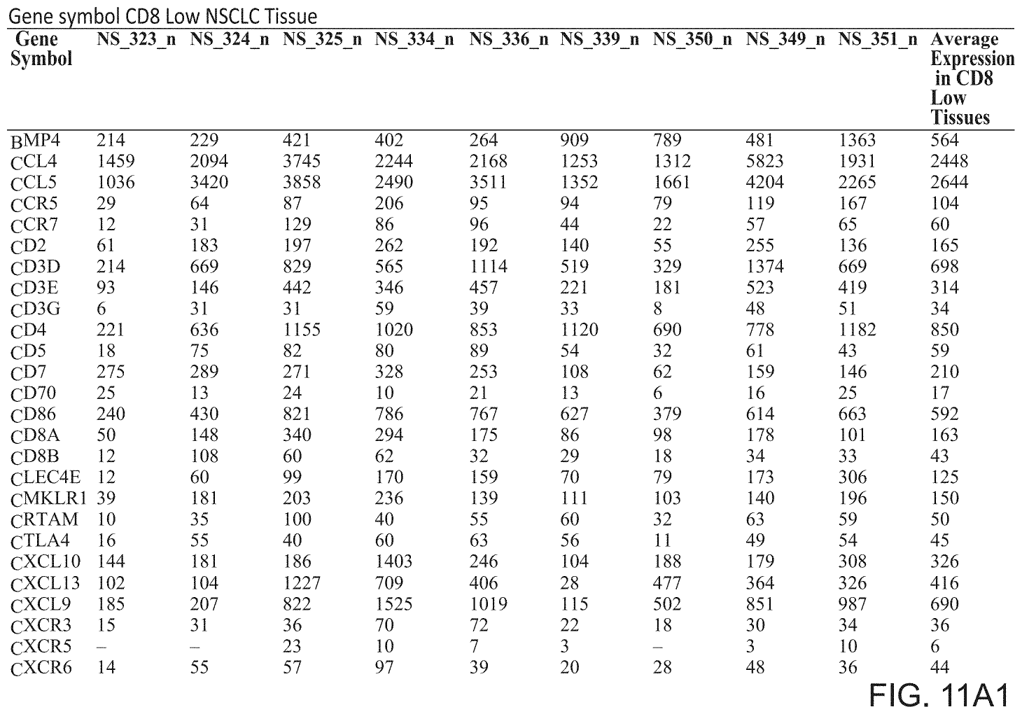

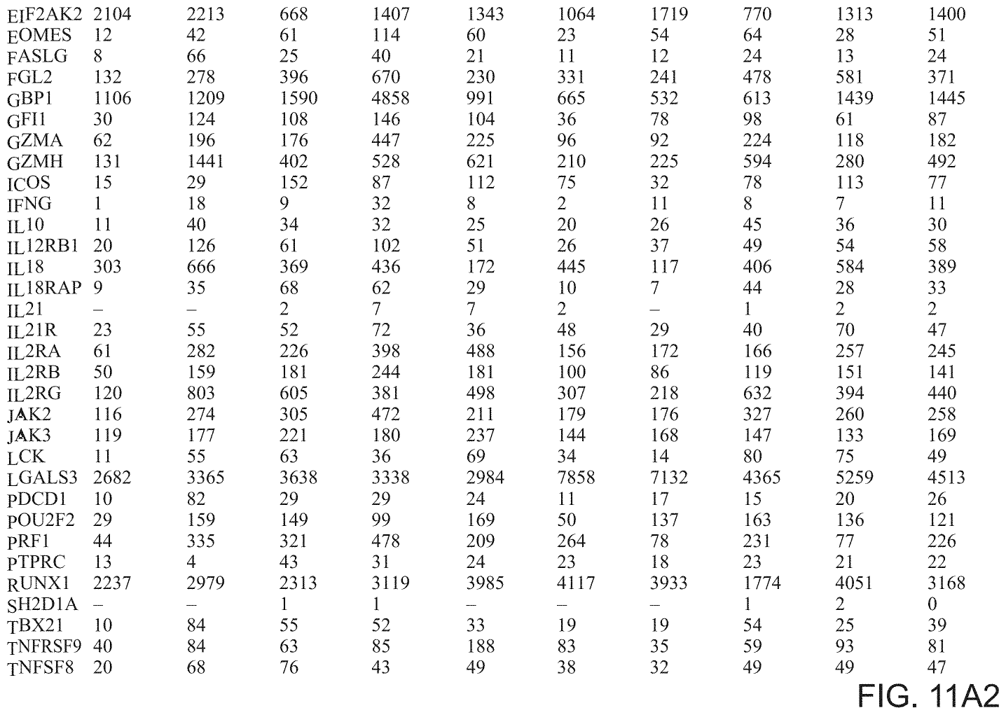

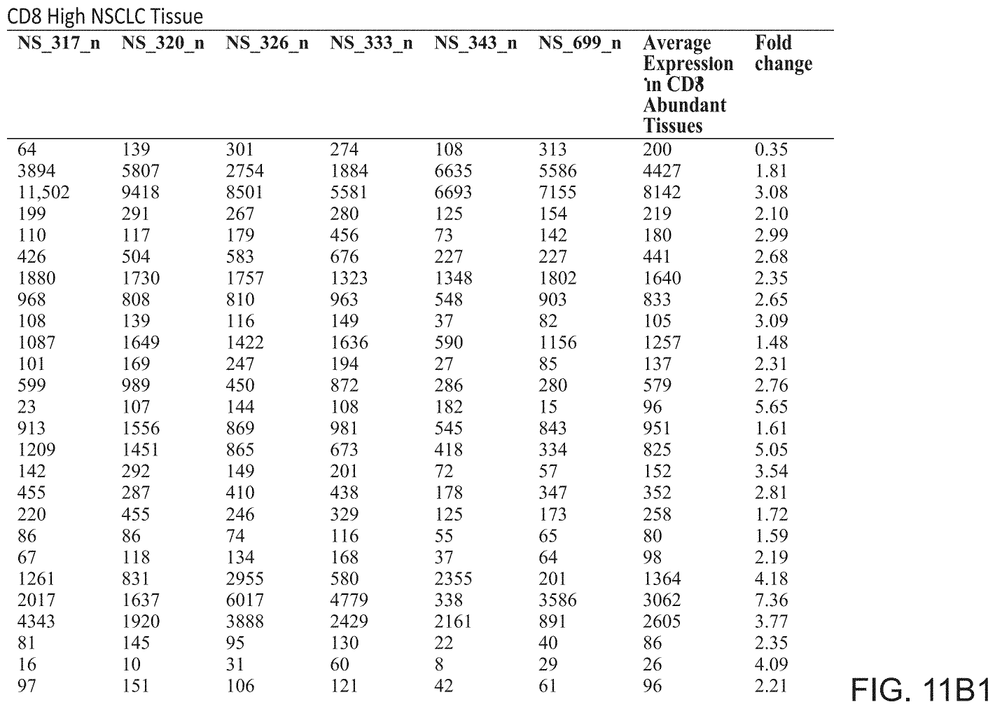

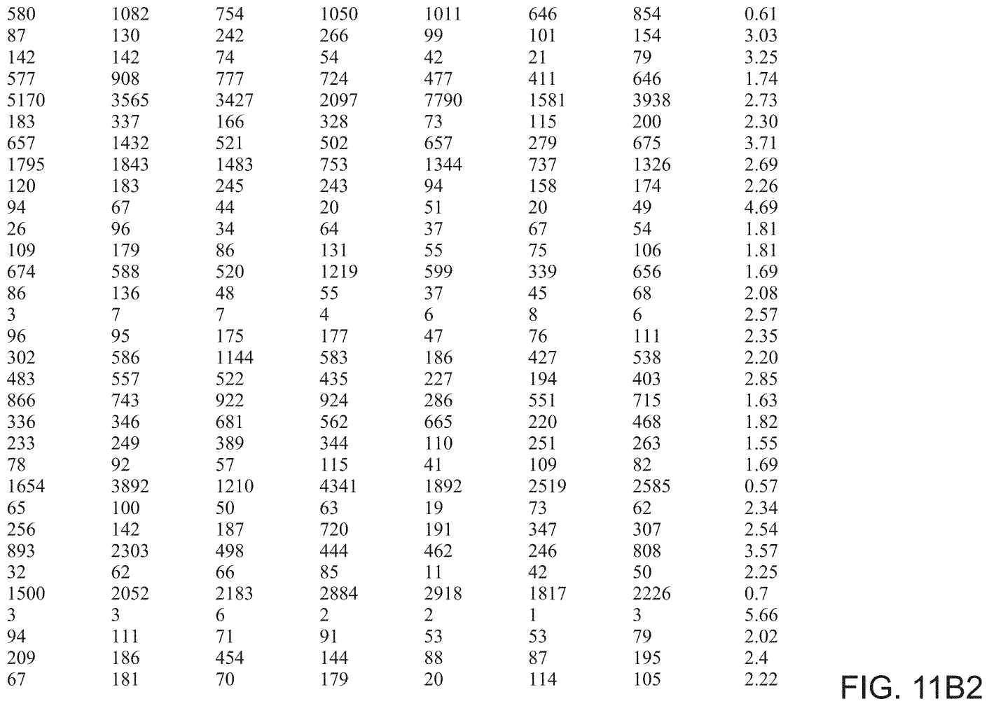

IDO1 is present in 10 tissues that also express moderate or high levels of PD-L1, and the distribution of IDO1 and PD-L1 varies within each NSCLC tissue. For example, PD-L1 is detected in the tumor and stroma of tissue NS_334, whereas IDO1 is solely expressed in the tumor (FIG. 6). Alternatively, PD-L1 is expressed in the tumor and surrounding stroma of tissue NS_1331, but IDO1 expression is limited to the surrounding stroma (FIG. 7). The variable expression of IDO1 and PD-L1 indicates that these that immune inhibitory proteins are expressed at unique levels within the tumor and stroma of NLSCC tissues.

Although the disclosure has been described and illustrated with a certain degree of particularity, it is understood that the present disclosure has been made only by way of example, and that numerous changes in the combination and arrangement of parts can be resorted to by those skilled in the art without departing from the scope of the disclosure, as hereinafter claimed.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified samples or steps and those that do not materially affect the basic and novel characteristic(s). Embodiments of the disclosure so claimed are inherently or expressly described and enabled herein.

Thus, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the disclosure.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. Method claims may be provided to present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary and Brief Description of the Drawings are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in any claim. Rather, as the following claims reflect, inventive subject matter lies in less than all features of a single disclosed configuration or operation.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A method for treating non-small cell lung cancer (NSCLC) in a subject in need thereof, the method comprising:
obtaining a biological sample from the NSCLC from the subject;
measuring the expression of the following genes associated with immune activation in the biological sample: IFNGR2, MICB, MICA, STATE, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, CCL2, CD28, CD40, OX40, 4-1BB, GITR, CD27, ICOS, CD226, B7, CD226, TCR, and CD40L;
measuring the expression of the following genes associated with immune inhibition in the biological sample: ES, CD86, HAVCR2, LAGS, PDCD1, TBX21, TNFRSF14, IDO1, PDCD1LG2, CD47, VTCN1, CD274, MIF, CD276, LGALS3, CTLA4, PD1, TIM3, BTLA, TIGIT, CD96, H3, VISTA, CD112R, and GITR;
determining that the NSCLC is responsive to an immunotherapy where the expression of fifteen or more of the genes associated with immune activation are upregulated and ten or less of the genes associated with immune inhibition are upregulated; and
administering an immunotherapy to the subject where the subject is determined to be responsive to the immunotherapy.

2. The method of claim 1, wherein the immunotherapy is an antibody.

3. The method of claim 2, wherein the antibody is a monoclonal antibody.

4. The method of claim 3, wherein the monoclonal antibody is an anti-PD-1 or anti-PD-L1 antibody.

5. The method of claim 4, wherein the antibody is pembrolizumab, nivolumab, or atezolizumab.

6. The method of claim 1, wherein the immunotherapy is a small molecule.

7. The method of claim 1, wherein the biological sample is assayed for genes associated with immune activation and genes associated with immune inhibition by analyzing nucleic acid obtained from the biological sample.

8. The method of claim 1, wherein the biological sample is assayed for genes associated with immune activation and genes associated with immune inhibition by analyzing protein obtained from the biological sample.

9. The method of claim 1, wherein the biological sample is from a tumor biopsy.

10. The method of claim 1, wherein the biological sample is from an aspirate.

11. A method for conducting a clinical trial by selecting subjects for the clinical trial that are responsive to an immunotherapy, the method comprising:
obtaining a biological sample from the NSCLC from the subject;
measuring the expression of the following genes associated with immune activation in the biological sample: IFNGR2, MICB, MICA, STATE, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, CCL2, CD28, CD40, OX40, 4-1BB, GITR, CD27, ICOS, CD226, B7, CD226, TCR, and CD40L;
measuring the expression of the following genes associated with immune inhibition in the biological sample: ES, CD86, HAVCR2, LAGS, PDCD1, TBX21, TNFRSF14, IDO1, PDCD1LG2, CD47, VTCN1, CD274, MIF, CD276, LGALS3, CTLA4, PD1, TIM3, BTLA, TIGIT, CD96, H3, VISTA, CD112R, and GITR;
determining that the NSCLC is responsive to an immunotherapy where the expression of fifteen or more of the genes associated with immune activation are upregulated and ten or less of the genes associated with immune inhibition are upregulated;
selecting subjects for inclusion in a clinical trial that are responsive to the immunotherapy;
administering an immunotherapy to the subject where the subject is determined to be responsive to the immunotherapy; and
seeking regulatory approval for the immunotherapy.

12. The method of claim 11, wherein the immunotherapy is an antibody.

13. The method of claim 12, wherein the antibody is a monoclonal antibody.

14. The method of claim 13, wherein the monoclonal antibody is an anti-PD-1 or anti-PD-L1 antibody.

15. The method of claim 14, wherein the antibody is pembrolizumab, nivolumab, or atezolizumab.

16. The method of claim 11, wherein the immunotherapy is a small molecule.

17. The method of claim 11, wherein the biological sample is assayed for genes associated with immune activation and genes associated with immune inhibition by analyzing nucleic acid obtained from the biological sample.

18. The method of claim 11, wherein the biological sample is assayed for genes associated with immune activation and genes associated with immune inhibition by analyzing protein obtained from the biological sample.

19. The method of claim 11, wherein the biological sample is from a tumor biopsy.

20. The method of claim 11, wherein the biological sample is from an aspirate.

21. A method for processing or analyzing a biological sample from a NSCLC from a subject, comprising:
(a) sequencing nucleic acid molecules from said sample of the NSCLC to yield data comprising one or more levels of gene expression products in said sample of the NSCLC, which one or more levels of gene expression products correspond to the following genes associated with immune activation: IFNGR2, MICB, MICA, STATE, IFIT1, IFIT2, IFIT3, IFNGR1, IL16, STAT4, STAT5A, STAT2, SOCS5, STAT3, TNFSF4, TNFRSF18, CXCL9, IFNG, STAT1, TNFRSF9, CXCL10, SOCS1, TNFRSF4, SOCS3, CCL2, CD28, CD40, OX40, 4-1BB, GITR, CD27, ICOS, CD226, B7, CD226, TCR, and CD40L, and the following genes associated with immune inhibition: ES, CD86, HAVCR2, LAGS, PDCD1, TBX21, TNFRSF14, IDO1, PDCD1 LG2, CD47, VTCN1, CD274, MIF, CD276, LGALS3, CTLA4, PD1, TIM3, BTLA, TIGIT, CD96, H3, VISTA, CD112R, and GITR;
(b) using an algorithm in a computer to process said data from (a) to generate a classification of said biological sample of NSCLC as responsive or unresponsive to an immunotherapy; and
(c) electronically outputting a report that identifies said classification of said sample of NSCLC as responsive or unresponsive to the immunotherapy.

22. The method of claim 21, wherein the immunotherapy is an antibody.

23. The method of claim 22, wherein the antibody is a monoclonal antibody.

24. The method of claim 23, wherein the monoclonal antibody is an anti-PD-1 or anti-PD-L1 antibody.

25. The method of claim 24, wherein the antibody is pembrolizumab, nivolumab, or atezolizumab.

26. The method of claim 21, wherein the immunotherapy is a small molecule.
